(19) United States
(12) Patent Application Publication
(10) Pub. No.: US 2026/0183015 A1
(43) Pub. Date: Jul. 2, 2026

(54) ROTATING EXPANDABLE BLADE DEBULKING TOOL HAVING A STATIONARY INNER SHAFT

(71) Applicant: KONINKLIJKE PHILIPS N.V., EINDHOVEN (NL)

(72) Inventors: Andrew SCHERER, MAPLE GROVE, MN (US); Ryan Michael SOTAK, COLORADO SPRINGS, CO (US)

(21) Appl. No.: 19/434,104

(22) Filed: Dec. 29, 2025

Related U.S. Application Data
(60) Provisional application No. 63/739,718, filed on Dec. 30, 2024.

(51) Int. Cl. A61B 17/3207

(52) U.S. Cl. CPC A61B 17/320725

(57) ABSTRACT

A vascular therapy device that includes an outer shaft, an inner shaft disposed coaxially within an outer shaft, and a blade assembly connected to the outer shaft and configured to rotate about the inner shaft along with the outer shaft.

### CROSS REFERENCE TO RELATED APPLICATIONS

This application claims the benefit of U.S. Provisional Patent Application Number 63/739,718 filed December 30, 2024. This application is hereby incorporated by reference herein.

### FIELD

The following relates generally to the catheter arts, intravascular therapy arts, lesion treatment arts, and related arts.

### BACKGROUND

Venous thromboembolism, which includes deep venous thrombosis (DVT), is a major contributor to the global disease burden and is the third most common cardiovascular pathology after coronary artery disease and stroke. Lower extremity DVT (LEDVT) can block the venous lumen and leads to venous congestion, swelling, and lower extremity venous valve function damage, resulting in post-thrombotic syndrome (PTS).

Standard treatment of venous obstruction includes the use of balloons, stents, lytics, aspiration and mechanical thrombectomy. Balloons and stents are inexpensive and time efficient treatment options but do not remove the obstruction from the vessel, which can lead to reoccurrence of the disease. Also, stents are typically not considered as a treatment option below the lesser trochanter due to poor long term patency in this anatomy. Lytics, aspiration, and mechanical thrombectomy treatments effectiveness drop significantly with the age of clot, becoming ineffective for chronic obstructions.

Another possible treatment includes a device that utilizes rotating blades to debulk the obstruction. In such a device, rotation of the blades can be performed via rotation of a hollow inner shaft or guidewire lumen. A possible disadvantage to this design is that rotating the inner shaft or guidewire lumen can cause unintended motion of the guidewire. Another possible disadvantage is that the inner shaft or guidewire lumen may be limited in the amount of torque it can generate.

The following discloses certain improvements to overcome these problems and others. Specifically, the following discloses embodiments wherein rotational blade motion is transferred from the handle to a distal end of the treatment device via an outer shaft instead of an inner shaft.

### SUMMARY

In accordance with one aspect of the present invention, a vascular therapy device is provided that includes an outer shaft, an inner shaft disposed coaxially within the outer shaft, and a blade assembly connected to the outer shaft and configured to rotate, along with the outer shaft, about the inner shaft.

In accordance with another aspect of the present invention, the outer shaft is configured to translate relative to the inner shaft along a longitudinal axis to selectively increase or decrease a radius of the blade assembly.

In accordance with another aspect of the present invention, the blade assembly includes a proximal end fixed to the outer shaft and a distal end that is inhibited from translating relative to the inner shaft in a longitudinal direction of the longitudinal axis.

In accordance with another aspect of the present invention, the vascular therapy device includes a keyed telescoping joint disposed between the proximal and distal ends of the blade assembly.

In accordance with another aspect of the present invention, the outer shaft includes a keyway that extends along a longitudinal axis of an outer shaft portion.

In accordance with another aspect of the present invention, the blade assembly includes a distal blade holder that includes a key that extends from the distal blade holder towards the longitudinal axis in a radial direction and is configured to engage with the keyway of the outer shaft portion.

In accordance with another aspect of the present invention, a retaining ring is disposed around the inner shaft and fixed thereto and is configured to inhibit longitudinal movement of the distal end of the blade assembly with respect to the inner shaft.

In accordance with another aspect of the present invention, a blade assembly includes at least one cutting blade oriented parallel to an outer shaft and an inner shaft and a longitudinal movement of the outer shaft in a distal direction relative to the inner shaft increases compressive bowing of the at least one cutting blade to expand the at least one cutting blade in a radial direction and longitudinal movement of the outer shaft in a proximal direction relative to the inner shaft decreases the compressive bowing of the at least one cutting blade to collapse the at least one cutting blade.

In accordance with another aspect of the present invention, a blade assembly for use in connection with catheter based vascular therapy is provided. The blade assembly includes a proximal end configured to be fixed to an outer shaft of a vascular therapy device such that the proximal end rotates and translates with the outer shaft with respect to a longitudinal axis of the blade assembly, a distal end configured to be fixed to an inner shaft of the vascular therapy device such that the distal end rotates about the inner shaft and translation along the longitudinal axis with respect to the inner shaft is inhibited, and a plurality of blades disposed between the proximal and distal ends.

In accordance with another aspect of the present invention, the blade assembly includes a keyed telescoping joint disposed between the proximal and distal ends.

In accordance with another aspect of the present invention, the blade assembly includes an outer shaft portion, and a distal blade holder, wherein relative motion between the outer shaft portion and the distal blade holder selectively increases and decreases a radius of the blades with respect to the longitudinal axis.

In accordance with another aspect of the present invention, the outer shaft portion includes a keyway disposed at the distal end of the outer shaft portion and the distal blade holder includes a key that extends radially from the distal blade holder towards the longitudinal axis and fits into the keyway.

In accordance with another aspect of the present invention, the blade assembly includes a plurality of blade alignment pieces and blade support pieces disposed on at least one of the proximal and distal ends.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a vascular therapy device in accordance with the present disclosure.

FIG. 2 diagrammatically illustrates a blade assembly in accordance with the present disclosure.

FIGS. 3A-3C diagrammatically illustrate embodiments of proximal end pieces in accordance with the present disclosure.

FIG. 3D diagrammatically illustrates a blade portion in accordance with the present disclosure.

FIG. 4 diagrammatically illustrates a distal end of the vascular therapy device in accordance with the present disclosure.

FIGS. 5A-5B diagrammatically illustrate a keyed telescoping joint in accordance with the present disclosure.

FIG. 6 diagrammatically illustrates a perspective view of a distal end portion of a vascular therapy device in accordance with the present disclosure.

FIG. 7 diagrammatically illustrates a distal end portion of a blade assembly in accordance with the present disclosure.

### DETAILED DESCRIPTION

The following relates to debulking tool designs for use in intravascular therapy. The tool has a blade assembly with a first end connected with an outer shaft and a second end connected at a distal end of the debulking tool. By manipulation of the outer shaft relative to an inner shaft, a radius and/or diameter of the blade assembly can be adjusted. Advantageously, this enables the radius to be adjusted during an intravascular procedure. For example, if there are two clots to be debulked at two different locations along a blood vessel, the vessel lumen size may be different at the two different locations. The radius/diameter of the blade assembly can be adjusted to conform with the vessel lumen at each location. As another advantage, the material being debulked can be processed in multiple passes, with the radial expansion of the blade assembly being increased with each pass, thereby debulking the clot in an annular layer-by-layer fashion. As yet another advantage, the adjustability of the blade assembly size enables a smaller number of such debulking tools to be stocked compared with the case of debulking tools whose cutting element radius/diameter is fixed, where different sizes may need to be kept in stock. As still yet another advantage, the size of the blade assembly can be reduced during insertion of the catheter to move the blade assembly to the treatment site.

In one design, the outer shaft is moved in a longitudinal direction relative to the inner shaft to expand or collapse the blade assembly. In this design the blade assembly is oriented longitudinally, i.e., parallel with the shafts, so it bows outward as the outer shaft is moved distally with respect to the inner shaft. The longitudinal movement of the outer shaft with respect to the inner shaft in this design could be driven by a motor or other translation mechanism disposed in a handle of the vascular therapy device.

The amount of longitudinal movement can be correlated with the radius or diameter of the blade assembly, so that the user can adjust the expansion of the blade assembly with knowledge (even apart from any imaging) of the expansion.

In variant embodiments, the cutting edges of the blade assembly could be serrated, and/or the radius/diameter of the blade assembly could be nonuniform over the length of the assembly.

The shafts are typically of stainless steel or the like, while the blades can be stainless steel, nitinol, or a shape memory polymer, for example. In addition, the blades can be cut from hypotubes.

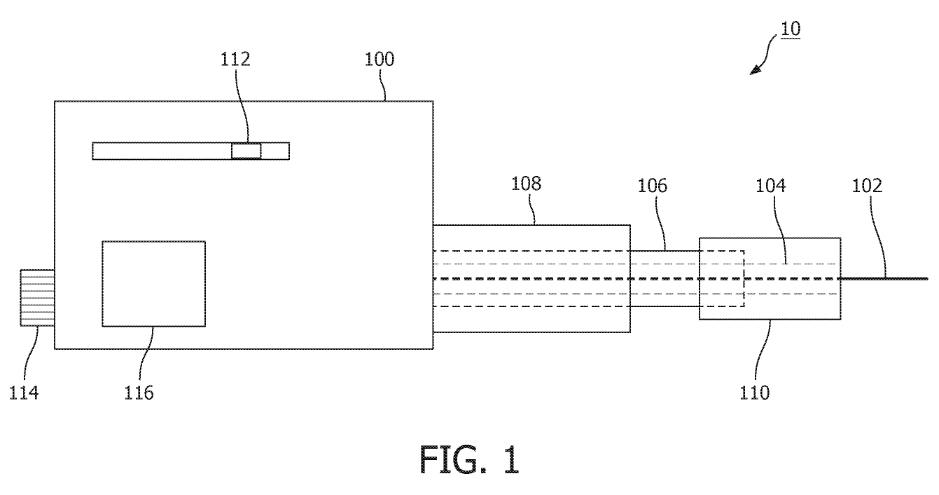

With reference to FIG. 1, an illustrative vascular therapy device 10 is diagrammatically shown. As shown in FIG. 1, the vascular therapy device 10 can be insertable along a guidewire 102 into a blood vessel for treating a lesion (or a clot, or an occlusion, and so forth) in the blood vessel. The vascular therapy device 10 includes, for example, a capture sheath 108, an outer shaft 106 disposed within the capture sheath 108, and an inner shaft 104 disposed coaxially within the outer shaft 106. In the embodiment shown, the inner shaft 104 extends distally from the distal end of the outer shaft 106. While the distal end of the capture sheath 108 is shown in a position that is proximal to the distal end of the outer shaft 106, it is to be understood that the capture sheath can be coupled to a slide mechanism 112 within handle 100. Slide mechanism 112 can be translated distally and proximally to extend and retract capture sheath over the outer shaft 108, inner shaft 104 and blade assembly 110 as desired.

Continuing with FIG. 1, a blade assembly 110 is connected to the outer shaft 106 at the proximal end of the blade assembly 110. As will be described in more detail below, the distal end of the blade assembly 110 is coupled to the distal end of the inner shaft 104 such that translation of the distal end of the blade assembly 110 in a longitudinal direction with respect to the inner shaft 104 is inhibited while allowing rotation of the blade assembly around the inner shaft 104. The outer shaft 106 is configured to move relative to the inner shaft 104 to expand or collapse the blade assembly 110. This movement in the longitudinal direction can be performed by a coupling of the outer shaft 106 to rotating knob 114 that can be disposed on the proximal end of handle 100. Rotating knob 114 can be coupled to outer shaft 106 via a threaded coupling piece (not shown) within the handle 100. As rotating knob 114 is rotated, outer shaft 106 can be translated proximally or distally as desired. As described in more detail below, as outer shaft 106 moves proximally or distally with respect to inner shaft 104, the blades of blade assembly 110 contract or expand radially as desired. In this regard, rotating knob 114 can be indexed or marked to indicate the amount of expansion of the blades. A motor 116 can also be disposed in handle 100. Motor 116 can be coupled to outer shaft 106 in order to impart rotation of the outer shaft 106 and blade assembly 110 about inner shaft 104 such that blades can engage material to be removed from a blood vessel.

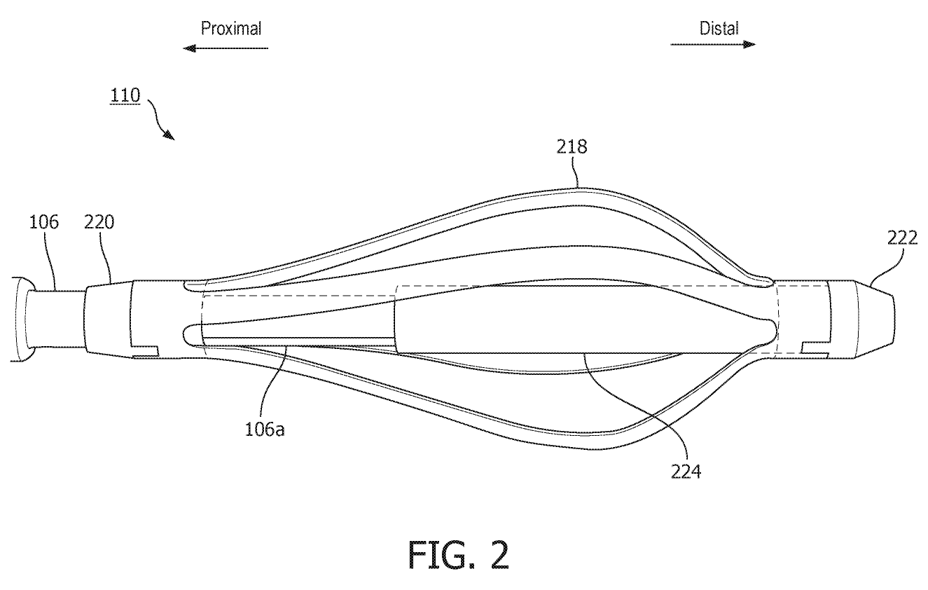

FIG. 2 shows an embodiment of blade assembly 110. In the embodiment shown, blade assembly 110 includes a proximal end piece 220, a distal end piece 222, and a blade portion 218 that extends between the proximal and distal end pieces 220, 222. In the embodiment shown, blade portion includes four blades distributed circumferentially around the longitudinal axis (not shown) of the blade assembly, but it is to be understood that one or more blades can be implemented as desired. It is also to be understood that the proximal end piece 220, blade portion 218, and distal end piece 222 could a unitary construction or assembled from separate pieces. As shown in FIG. 2, the outer shaft 106 runs through the proximal end of the blade assembly and into a distal blade holder 224. It is to be understood, however, that outer shaft portion 106a could be an extension of outer shaft 106 or it could be a separate piece that is attached, along with the blade assembly 110, to the outer shaft 106. As described more fully below, the coupling of outer shaft portion 106a and distal blade holder 224 can be made via a keyed telescoping joint. The keyed telescoping joint facilitates the expansion and contraction of the blade portion as the outer shaft portion 106a is moved distally and proximally within distal blade holder 224. As shown, blade portion 218 is in an expanded position. It is to be understood that in a contracted position, the individual blades of blade portion 218 run substantially parallel to the longitudinal axis of the blade assembly 110.

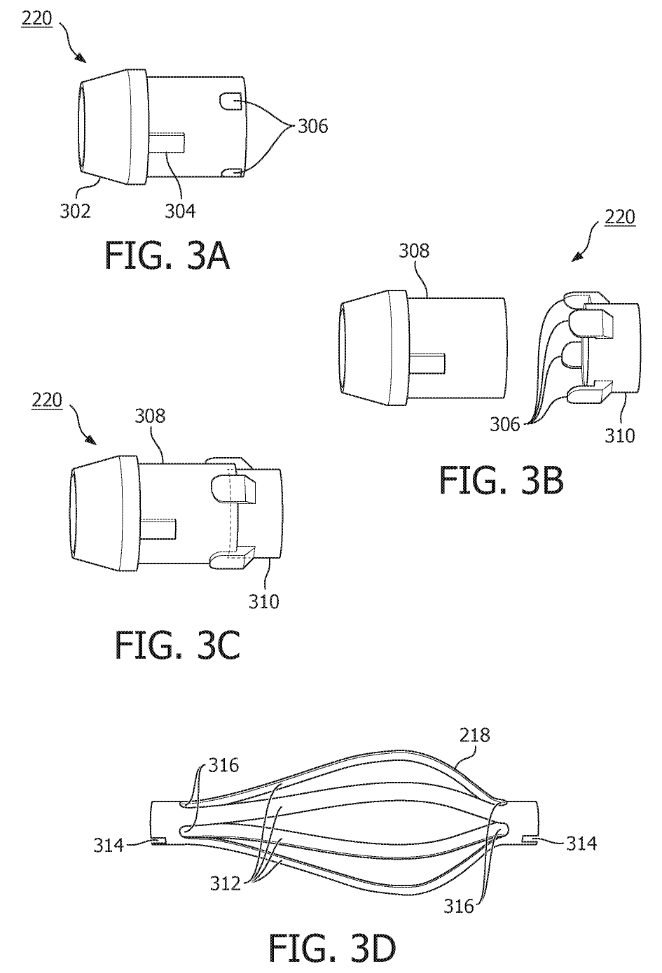

FIGS. 3A-3D show embodiments of the proximal end piece 220 and blade portion 218. In FIG. 3A, proximal end piece includes a proximal tip piece 302, a proximal blade alignment piece 304 that extends radially and distally from the proximal tip piece 302, and proximal blade support pieces 306 that extend radially from the proximal end piece 220. In the embodiment of FIG. 3A, proximal end piece 220 is shown as being a unitary construction.

FIGS. 3B and 3C show an embodiment of the proximal end piece 220 that has a proximal portion 308 and distal portion 310 that are separate pieces that can be joined together as shown in FIG. 3C. FIG. 3D shows a blade portion having four separate blades 312. It is to be understood that the blade portion is not limited to having four blades and that fewer or more blades are contemplated. In an embodiment where blade assembly 110 includes proximal end piece 220 and blade portion 218, when proximal end piece 210 and blade portion are coupled, proximal blade alignment piece 304 fits into the alignment notch 314 that is disposed at the proximal end of blade portion 218. In addition, proximal blade support pieces 306 fit in between blades 312 at blade support notches 316 that are formed between the proximal ends of adjacent blades. Regardless of the component parts, once the proximal end of the blade assembly 110 are assembled, the components are secured together and fixed to the outer shaft 106 so that rotation of the outer shaft 106 imparts rotation to the blade assembly 110 while limiting torque and/or energy loss through the system. Such fixation can be done through welding, gluing, press fitting, and the like. It is to be understood that the distal end of the blade portion 218 can be similarly joined to distal end piece 222 and distal blade holder 224.

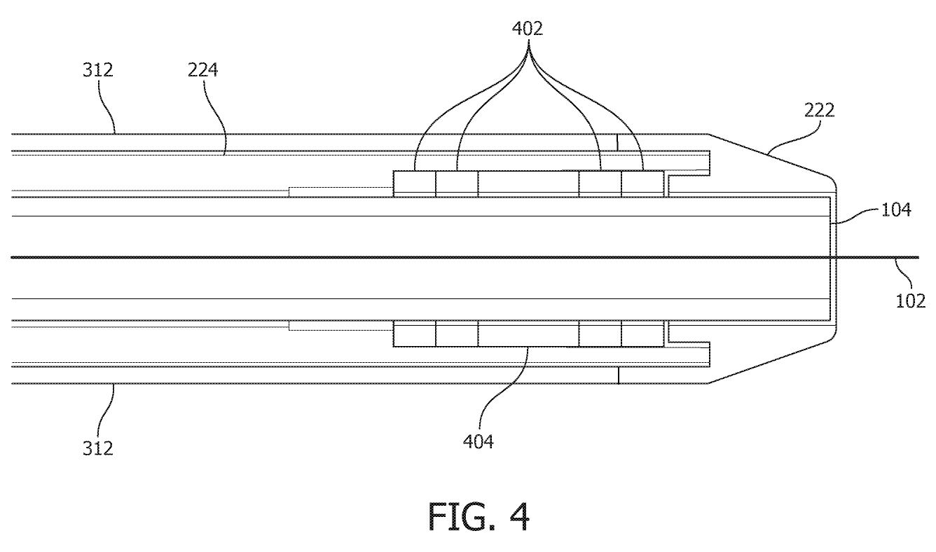

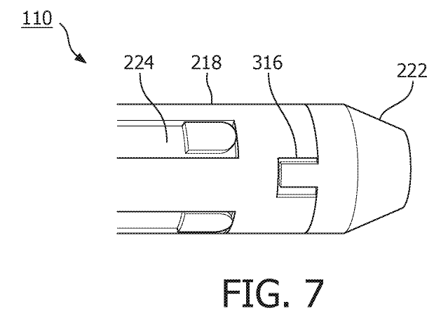

FIG. 4 shows a cross sectional view of the distal end of the vascular therapy device. As shown, two cutting blades 312 (in a radially compressed position) can be seen; one on the top of the device and one on the bottom of the device. Distal blade holder 224 is disposed between inner shaft 104 and blades 312 and fits into distal end piece 222. As shown in FIG. 7, the distal end of the blade portion 218 can fit onto the distal blade holder 224 and distal end piece 222 analogously to the way in which the proximal end of the blade portion 218 fits onto proximal end piece 210. As noted above, the distal end of blade portion 218, the distal blade holder 224, and the distal end piece 222 can be secured to one another by welding, glue, or other fixation, such that they rotate about the inner shaft 104 when the outer shaft is rotated about the inner shaft.

Also shown in FIG. 4 is a retaining ring 404 disposed around the inner shaft 104. This retaining ring 404 is positioned between the distal blade holder 224 and distal end piece 222 such that retaining ring 404 inhibits longitudinal motion of the distal end of the blade assembly 110 relative to the inner shaft 104. In one embodiment, retaining ring 404 can be welded, or otherwise affixed to inner shaft 104. Further, in constructing a rotating joint around the inner shaft 104, washers 402 can be disposed around inner shaft 104 on the proximal and/or distal sides of retaining ring 404. As shown, there are four washers disposed around inner shaft 104, but it is to be understood that various numbers of washers could be used. Washers 402 can improve wear properties of the rotating joint and increase longevity. In one embodiment, 304 stainless steel can be used for the retaining ring 404 and 17-4 PH material can be used for the distal blade holder 224 and distal end piece 222. Washers 402 can be implemented using 440 stainless steel washers in a full hard condition.

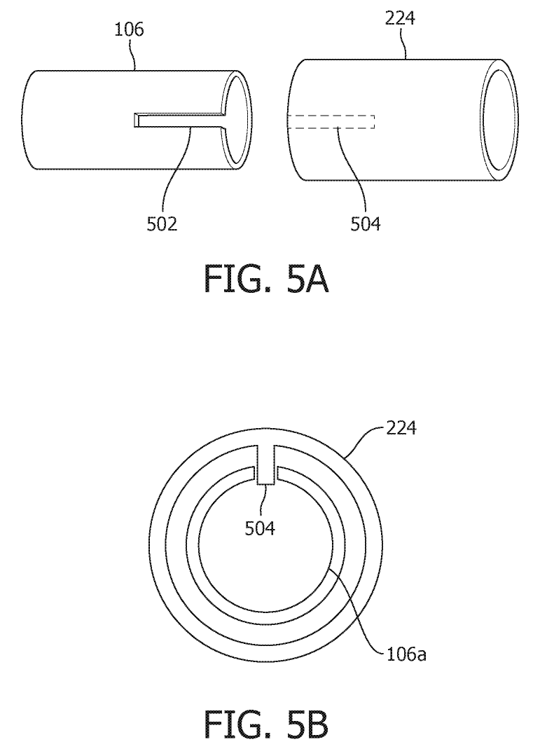

FIGS. 5A-5B show how outer shaft 106 and distal blade holder 224 can be configured to form a keyed telescoping joint. As shown in FIG. 5A, outer shaft 106 can include a keyway 502 that is cut or otherwise formed in a distal end of outer shaft portion 106a. Distal blade holder 224 is shown to include a compatible key 504 that extends radially towards the longitudinal axis of distal blade holder 224. While FIG. 5A shows outer shaft portion 106a and distal blade holder 224 as being separated, in operation, the distal end of outer shaft portion 106a and proximal end of distal blade holder 224 overlap each other such that key 504 is disposed within keyway 502 as shown in FIG. 5B. The longitudinal lengths of keyway 502 and key 504 can be long enough to accommodate a desired motion of the outer shaft 106 with respect to the distal blade holder 224 in a longitudinal direction. More specifically, the respective longitudinal lengths can be selected such that when outer shaft 106 is in a most proximal longitudinal position with respect to distal blade holder 224, blades 312 are in a most extended (or flat) position and when outer shaft 106 is in a most distal position with respect to distal blade holder 224, blades 312 are in a most radially expanded position. Alternately, while not specifically shown, distal blade holder 224 could include a keyway and outer shaft portion 106a could include a key as analogously described above without deviating from the intended purposes or functions of embodiments of the inventions described herein.

While FIG. 5B shows spaces between outer shaft 106 and distal blade holder 224, it is to be understood that respective inner and outer diameters of outer shaft portion 106a, distal blade holder 224, and inner shaft 104 are dimensions to inhibit motions of these components with respect to each other in the radial direction while allowing relative motion between the these components in the longitudinal and rotational directions as described herein. In addition, keyway 502 and key 504 are dimensioned to allow longitudinal motion of outer shaft 106 with respect to distal blade holder 224 while inhibiting rotational movement of these components with respect to each other. In such a design, blades 312 can be selectively expanded and collapsed as desired and rotational torque applied to outer shaft 106 can be transmitted effectively to the proximal and distal ends of blade assembly 110. In other words, the keyed telescoping joint facilitates rotational torque applied to outer shaft 106 to be transmitted to the distal end of blade assembly while inhibiting twisting of the proximal and distal ends of blade assembly 110 with respect to each other around the longitudinal axis.

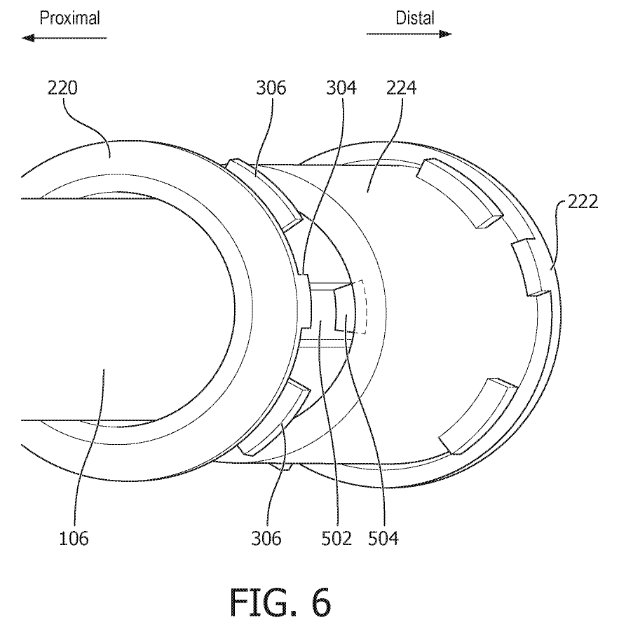

FIG. 6 shows a perspective view of the blade assembly without blade portion 218. As can be seen, outer shaft 106, including keyway 502 in outer shaft portion 106a, is in a longitudinally slidable connection with distal blade holder 224. Key 504 of distal blade holder 224 extends radially into keyway 502 to provide rotational stability and longitudinal alignment of outer shaft 106 with respect to distal blade holder 224. Proximal end piece 224 of blade assembly 110 is shown with outer shaft 106 passing therethrough. Proximal blade alignment piece 304 and proximal blade support pieces 306 are shown on proximal end piece 220. At the distal end of FIG. 6, distal end piece 222 is shown along with distal blade support and alignment pieces. FIG. 7 shows the interconnection of the distal end of blade portion 218, the distal end of distal blade holder 224, and distal end piece 222.

While blade assembly 110 has been described as having various separate components, it is to be understood that the assembly can be constructed from various components or as a unitary construction provided that its proximal and distal ends are configured to move longitudinally with respect to one another while rotational movement around its longitudinal axis with respect to each other is inhibited. As noted above, this construction can be accomplished by implementing a blade assembly that has an outer shaft portion 106a that mates with a distal blade holder 224 via a keyed telescoping joint 502, 504. In this regard, outer shaft portion 106a can be an extension of outer shaft 106 or it can be separate from outer shaft and blade assembly 110 can be attached to outer shaft 106 as otherwise described herein.

In operation, vascular therapy device 10, can be delivered over a guidewire 102 to a region of interest of a subject. During delivery, blade assembly 110 can be in a contracted or compressed state such that the blades 312 run generally parallel with a longitudinal axis of vascular therapy device 10. During delivery, blade assembly 110 can be delivered inside an outer, or capture sheath, 108 to avoid contact of the blade assembly with a blood vessel during delivery. Once positioned, the vascular therapy device 10 then could be deployed out of the capture sheath, and the knob 114 on the handle 110 can be turned to adjust the effective radius or diameter of the blade assembly 110 until it is sized appropriately for the anatomy being treated. Once the treatment size is selected, blade assembly 110 can be rotated to debulk the obstruction. Imaging such as x-ray imaging, intravascular ultrasound (IVUS), extravascular ultrasound (EVUS), etc. can optionally be used to assess the degree of debulking achieved and guide further treatment. Once the desired amount of material is debulked, the blade assembly 110 can be collapsed, returned to the capture sheath, and pulled back into the catheter and removed from the subject.

Rotation of the blade assembly is performed by rotating the outer shaft 106 about its longitudinal axis and around the inner shaft 104. The inner shaft 104 can extend from handle 100 of the vascular therapy device to a distal end of the vascular therapy device. Rotation of the inner shaft 104 about its longitudinal axis is limited by fixing the inner shaft to the handle so that rotation and translation of the inner shaft 104 relative to the handle is limited. In one embodiment, the inner shaft extends from the distal tip of the vascular therapy device to a proximal luer fitting in the handle of the device. The distal end of the blade assembly is attached to the distal end of the inner shaft via a swivel or rotational joint. This allows the blade assembly to rotate independently of the inner shaft, while allowing the inner shaft to hold the longitudinal location of the distal end of the blade assembly stationary relative to the inner shaft. The rotary motion of the blade assembly is then transferred from the handle by the outer shaft to the proximal end of the blade assembly at it is rigidly attached to the outer shaft. The outer shaft can translate longitudinally via controls on the handle to move the proximal end of the blade assembly longitudinally to control the radius/diameter of the blade portion. Rotation from the proximal end of the blade assembly is also transferred to the distal end of the blade assembly via a keyed telescoping joint. This ensures that the proximal end of the blade assembly rotates in unison with the distal end of the blade assembly, so that axial twisting in the blade assembly is limited. The keyed telescoping joint transfers this synchronized rotational movement from the proximal end to the distal end of the blade assembly, but still allows the proximal end of the blade assembly to move longitudinally relative to the distal end of the blade assembly so that the blades can open and close radially.

To further tailor the performance of the device to a specific anatomy or disease state, the geometry of the blades can be varied to make the device more or less aggressive to match the needs of the situation. The blades can be designed with one side having a smooth or rounded edge, and the other side being sharp and/or serrated. In this way, when a more aggressive treatment is needed the device can be rotated so that the sharp/serrated edge of the blade is leading, and when a less aggressive treatment is needed the device can be rotated in the opposite direction. In this regard, motor can have a switch to select the rotation direction of the device so the operator can switch back and forth between the modalities during the procedure as desired.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A vascular therapy device, comprising:
an outer shaft;
an inner shaft disposed coaxially within the outer shaft; and
a blade assembly connected to the outer shaft and configured to rotate about the inner shaft along with the outer shaft.

2. The vascular therapy device of claim 1, wherein the outer shaft is configured to translate relative to the inner shaft along a longitudinal axis to selectively increase or decrease a radius of the blade assembly.

3. The vascular therapy device of claim 1, wherein the blade assembly includes a proximal end and a distal end and wherein the proximal end is fixed to the outer shaft and the distal end is inhibited from translating in a longitudinal direction of the longitudinal axis with respect to the inner shaft.

4. The vascular therapy device of claim 1, wherein the vascular therapy device further comprises a keyed telescoping joint disposed between the proximal and distal ends of the blade assembly.

5. The vascular therapy device of claim 4, further comprising an outer shaft portion that includes a keyway that extends along a longitudinal axis of the outer shaft portion.

6. The vascular therapy device of claim 4, wherein the blade assembly includes distal blade holder that includes a key that extends from the distal blade holder towards the longitudinal axis in a radial direction and is configured to engage with the keyway of the outer shaft portion.

7. The vascular therapy device of claim 1, further comprising a retaining ring disposed around the inner shaft and fixed thereto and configured to inhibit longitudinal movement of the distal end of the blade assembly with respect to the inner shaft.

8. The vascular therapy device of claim 1, wherein the blade assembly comprises at least one cutting blade oriented parallel to the outer shaft and the inner shaft and a longitudinal movement of the outer shaft in a distal direction relative to the inner shaft increases compressive bowing of the at least one cutting blade to expand the at least one cutting blade in a radial direction and longitudinal movement of the outer shaft in a proximal direction relative to the inner shaft decreases the compressive bowing of the at least one cutting blade to collapse the at least one cutting blade.

9. A blade assembly for use in connection with catheter bases vascular therapy, the blade assembly comprising:
a proximal end configured to be fixed to an outer shaft of a vascular therapy device such that the proximal end rotates and translates with the outer shaft with respect to a longitudinal axis of the blade assembly;
a distal end configured to be fixed to an inner shaft of the vascular therapy device such that the distal end rotates about the inner shaft and translation along the longitudinal axis with respect to the inner shaft is inhibited; and
a plurality of blades disposed between the proximal and distal ends.

10. The blade assembly of claim 9, further comprising a keyed telescoping joint disposed between the proximal and distal ends.

11. The blade assembly of claim 9, further comprising:
an outer shaft portion; and
a distal blade holder, wherein relative motion between the outer shaft portion and
the distal blade holder selectively increases and decreases a radius of the blades with respect to the longitudinal axis.

12. The blade assembly of claim 11, wherein the outer shaft portion includes a keyway disposed at a distal portion of the outer shaft portion and the distal blade holder includes a key that extends radially from the distal blade holder towards the longitudinal axis and fits into the keyway.

13. The blade assembly of claim 9, further comprising:
a plurality of blade alignment pieces and blade support pieces disposed on at least one of the proximal and distal ends.
